(19) United States
(12) Patent Application Publication
(10) Pub. No.: US 2025/0138022 A1
(43) Pub. Date: May 1, 2025

(54) SYSTEMS AND METHODS FOR IMPROVED METABOLOMICS

(71) Applicant: Ai-Genetika Inc., doing business as BioTwin, Québec (CA)

(72) Inventors: Louis-Philippe NOEL, Québec (CA); Camille Houle, Québec (QC); Manon FRADIN, Québec (CA); Frédéric AUDET, Québec (CA); Caroline RHÉAUME, Québec (QC)

(73) Assignee: Ai-Genetika Inc., doing business as BioTwin, Québec (CA)

(21) Appl. No.: 18/498,746

(22) Filed: Oct. 31, 2023

(51) Int. Cl. G01N 33/68; A61B 5/15

(52) U.S. Cl. CPC G01N 33/6848; A61B 5/15; G01N 2800/04

(57) ABSTRACT

A system for non-targeted analysis of a metabolome comprises a sample receiving area for receiving a sample from a subject, at least one mass spectrometer and means for further performing at least one of spectroscopy, high-throughput mass spectrometry, and nuclear magnetic resonance (NMR) on the sample, being operatively connected to the sample receiving area for receiving at least a portion of the sample therefrom. The at least one mass spectrometry and at least one of spectroscopy, high-throughput mass spectrometry and NMR identify a plurality of metabolites in the sample corresponding to at least a portion of the subject's metabolome. Non-targeted analysis yields a broad metabolomic profile including at least a portion of the subject's dark metabolome. The metabolomic analysis can be used to personalize wellness regimes and treatment.

### TECHNICAL FIELD

The present disclosure pertains to the field of analytical techniques. More specifically, the present disclosure pertains to metabolomics, particularly to analytical techniques useful for building metabolomic profiles and/or exploring the dark metabolome using a non-targeted approach on biological samples.

### BACKGROUND

A patient's individual metabolome provides valuable insights into the person's health. For example, the presence of certain metabolites, or changes in their concentration in a fluid or tissue, may indicate beneficial or malignant trends or conditions before the patient exhibits any symptoms.

Growing pressure on health systems, both public and private, brings increased attention to preventative medicine. Effective personalized preventative medicine requires numerous patient monitoring activities, such as frequent check-ups and lab tests, that exceed current lab and physician capacity.

Personalizing healthcare through precision medicine can improve patient outcomes by identifying optimal treatment plans, anticipating and preventing serious treatment side-effects and tailoring monitoring programs to reduce unnecessary labwork.

Blood analysis is relied on by physicians and other health professionals to provide information about a patient's health. Bloodwork may form part of a regular annual check-up, or it may be requisitioned ad-hoc. Current blood analysis relies on a supply of liquid venous blood for testing. Accordingly, blood draws typically involve the services of a nurse or other licensed health professional, and may require drawing four or more vials. This is both unpleasant and time-consuming for the patient, as well as a significant use of healthcare resources. Inventory challenges may delay or prevent scheduled blood draws from proceeding. Furthermore, currently available low-throughput techniques mean that results may not be available for several days and that lab capacity acts as a bottleneck.

Non-invasive urine analysis is currently used to detect or monitor a range of medical conditions in the liver, kidneys and the urinary tract, as well as diabetes. Current techniques target the detection and measurement of known compounds in urine.

Use of salivary analysis is not currently widespread in the health system, except as a means for collecting patient DNA for sequencing. The scope of other screenings using salivary analysis remains experimental and limited, and health professionals are generally not inclined to consider salivary analysis.

There is therefore a need for analytical systems, individual monitoring systems, and methods therefor and that provide both ample and precise information about a patient's health and wellness while remaining easy to comply with.

### SUMMARY

According to a broad aspect, a system for non-targeted analysis of a metabolome in at least one sample comprises a sample receiving area for receiving at least one sample from a subject, at least one mass spectrometer for performing at least one mass spectrometry, operatively connected to the sample receiving area for receiving at least a portion of the at least one sample therefrom, and means to further perform at least one of spectroscopy, high-throughput mass spectrometry, and nuclear magnetic resonance (NMR), operatively connected to the sample receiving area for receiving at least a portion of the at least one sample therefrom, the at least one mass spectrometry and at least one of spectroscopy, high-throughput mass spectrometry and NMR identifying a plurality of metabolites corresponding to the subject's metabolome.

In embodiments, the at least one sample is provided by a subject, and the system is configured to output a metabolomic profile of the subject comprising all detected metabolites. In embodiments, the system is configured to analyze a plurality of samples, in parallel and/or in sequence.

In embodiments, the at least one sample is provided on an absorbent substrate.

In embodiments, the system further comprises mechanical separation means for mechanically separating at least a portion of the absorbent substrate.

In embodiments, the system further comprises solvent provision means for providing a solvent to extract, solubilize, dissolve, or dilute at least a portion of the at least one sample.

In embodiments, the at least one sample comprises a urine sample, a blood sample, and/or a saliva sample. In embodiments, the blood sample is a capillary blood sample.

According to a broad aspect, a synergistic metabolomic analytical method for assessing a subject's health or wellness, the method comprises providing at least one sample from the subject, performing untargeted mass spectrometry on at least a portion of the at least one sample, performing at least one untargeted analytical technique selected from the group consisting of: spectroscopy, NMR, another mass spectrometry on the sample, and outputting a metabolomic profile for the subject.

In embodiments, the outputting the metabolomic profile comprises outputting a dark metabolome profile for the subject.

In embodiments, the method further comprises storing at least one result from each of the untargeted mass spectrometry and the untargeted analytical technique in a non-transitory storage medium and applying a Machine Learning Algorithm to the at least one result so stored.

In embodiments, the method further comprises issuing, to the subject, based on the application of the Machine Learning Algorithm to the at least one result, a recommendation with respect to at least one of nutrition, exercise, prescription medications and an appointment with a physician.

According to a broad aspect, a kit for collecting at least one sample from a subject comprises at least one absorbent substrate, the substrate being configured to receive the sample from the subject, storage means for separately storing each absorbent substrate having received the sample, and an envelope configured to receive the storage means, the envelope meeting requirements for biological or medical material mailing.

In embodiments, the kit further comprises at least one of blood drawing means and disinfection means. In embodiments, the blood drawing means is a sterile lancet. In embodiments, the disinfection means comprise at least one disinfectant wipe.

The present disclosure provides systems and methods for global and/or holistic profiling of a subject's metabolome.

The present disclosure provides systems and methods for global and/or holistic profiling of a subject's metabolome.

### BRIEF DESCRIPTION OF THE DRAWINGS

Having thus generally described the nature of the invention, reference will now be made to the accompanying drawings, showing by way of illustration example embodiments thereof and in which:

FIG. 1 shows an exemplary system for analyzing a biological sample in accordance with the present disclosure.

FIG. 2 shows an exemplary process for analyzing a biological sample in accordance with the present disclosure comprising a plurality of analytical techniques, such as mass spectrometry analysis.

FIG. 3 shows an exemplary process for analyzing a biological sample in accordance with the present disclosure comprising mass spectroscopy and Nuclear Magnetic Resonance (NMR) analysis.

FIG. 4 shows an exemplary kit for collecting at least one sample from a subject according to the present disclosure.

### DETAILED DESCRIPTION

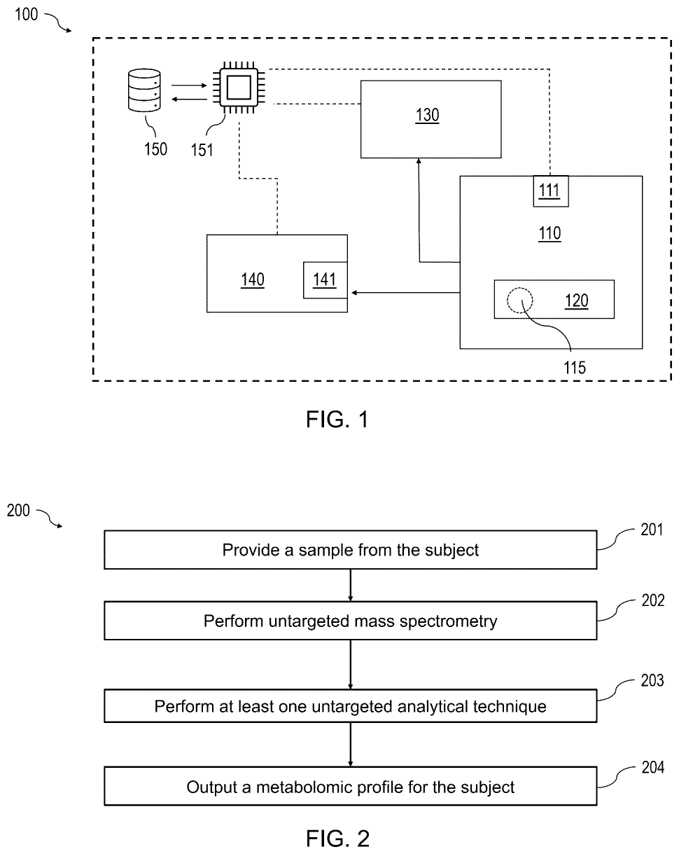

Referring to FIG. 1, an exemplary system 100 for the non-targeted analysis of a sample comprises a sample receiving area 110. The sample receiving area 110 is configured to receive a sample 115, for example a biological sample obtained from a subject. The sample 115 may be a biological sample suitable for metabolomic analysis, such as, but not limited to, a blood sample, a urine sample, a saliva sample. It will be understood that other biological fluids and biosamples may be collected and provided to the system 100 without departing from the present teachings.

The sample receiving area 110 may be suitably configured to receive a sample 115. For example, the sample receiving area 110 may define one or more cavities, openings, grooves or pockets apt to receive a sample 115. For example, in a non-limiting embodiment, the sample receiving area 110 may define an opening comprising sample holding means, for example clips or other means for holding an object in place. A user places a sample 115 in the sample receiving area 110 for the system 100 to process and analyze the sample as described below.

The sample receiving area 110 may comprise means to prepare the sample 115 for processing and analysis. For example, the sample receiving area 110 may comprise extraction, solubilization or dissolution means, for example means for delivering a quantity of solvent, of water, of an acid, of a base to the sample 115. The solvent may be aqueous and may comprise one or more organic compounds. For example, the sample 115 may comprise an absorbent substrate 120 on which a quantity of fluid, such as blood, urine, or saliva has been absorbed. The system 100 may provide a solvent, such as an alcohol, or water, to at least a portion of the absorbent substrate 120, thereby allowing the analytes such as metabolites, proteins, or lipids to be extracted, solubilized, desorbed and/or dissolved from the absorbent substrate 120.

The sample 115 may be provided to the system 100 on an appropriate substrate. For example, the sample 115 may be provided to the system 100 on an absorbent substrate 120. The absorbent substrate 120 may, in a non-limiting embodiment, be a strip of absorbent paper configured to receive one or more samples from a subject.

In another non-limiting embodiment, the absorbent substrate 120 may be configured to receive one or a plurality of samples within the same sampling collection session. For example, the absorbent substrate 120 may be configured to receive one or more blood blots, or streaks, or other form of blood sample. In such an example, a subject's finger or other suitable part of the body may be pierced, pricked, lanced, or otherwise caused to bleed in amounts appropriate for collecting a blood streak, blot, or other appropriate blood sample. It is understood that the absorbent substrate 120 may be configured to receive the plurality of samples in generally rapid succession, for example as three blood dots collected sequentially within a few seconds of each other. The absorbent substrate 120 may be configured to receive a plurality of samples at different times. For example, the absorbent substrate 120 may be configured to receive one or more samples such as blood dots before the subject has a meal and one or more of said samples after the subject has eaten. Appropriate preserving means for preserving the quality and characteristics of the samples will be apparent to a skilled person.

The absorbent substrate 120 may be then placed in contact with the finger or body part so caused to bleed, and at least a quantity of the blood may form one or more blots, dots, or streaks on the absorbent substrate 120. Other collection methods may also be appropriate. For example, the subject may be instructed to cause one or more drops of blood to drip by gravity onto the absorbent substrate 120, thereby limiting skin-to-substrate cross-contamination. In a non-limiting embodiment, the absorbent substrate 120 may be configured to receive up to four blood dots, whereby the subject's finger of body part, having been caused to bleed, is successively placed in contact with four distinct portions of the absorbent substrate 120 or is held above the absorbent substrate 120 to cause one or more drops of blood to fall onto the absorbent substrate 120, the distinct portions being appropriately spaced for receiving four distinct blood dots substantially separate from each other.

The distinct portions may be marked with appropriate indicia, such as printed indicia printed on the absorbent substrate 120. The printed indicia may comprise shapes and/or other characters, such as numbers or letters. For instance, the printed indicia may be separate circles marked with a number or a letter in proximity to each circle, directing or suggesting to a user, such as a subject or a healthcare professional, an order in which the distinct portions of the absorbent substrate 120 may be placed into contact with the finger or body part.

For example, the absorbent substrate 120 may be configured to receive a urine sample. It will be understood that the absorbent substrate 120 is suitable for receiving the sample and convenient to manipulate. For example, an absorbent substrate 120 configured to receive a urine sample may be attached to an elongated strip or paper, of resilient material such as plastic, or another elongated object, thereby allowing a subject or a healthcare professional to collect a urine sample conveniently. It is understood that the absorbent substrate 120 may be appropriately resilient and/or rigid without the use of additional resilience and/or handling means. For example, the absorbent substrate 120 may comprise appropriately stiff paper, cardboard, fibers and/or fabrics, or stiffness additives as will be apparent to a skilled person.

In another non-limiting embodiment, the absorbent substrate 120 may be configured to receive a saliva sample. The absorbent substrate 120 so configured may be provided with appropriate collection and manipulation means. For example, in a non-limiting embodiment, the absorbent substrate 120 configured to receive a saliva sample may be provided with a resilient and/or a non-absorbent portion for more convenient manipulation during sample collection. The absorbent substrate 120 may be configured to be held by a subject at one end and used for collecting saliva at another end without the use of additional manipulation means.

The system 100 comprises means for carrying out at least one mass spectrometry analysis, for example a mass spectrometer 130. The mass spectrometer 130 may be of any acceptable type, for example a quadrupole-time-of-flight (QToF), triple quadrupole (QqQ), MALDI time-of-flight (MALDI-ToF), orbitrap.

In some non-limiting embodiments, the sample-receiving area 110 may comprise a puncher (not shown) configured to punch out portions of the absorbent substrate 120, wherein the portions so punched out may be collected automatically and prepared for analysis. For example, the system 100 may be configured to receive a sample 115 on an absorbent substrate 120, punch out one or more portions, wherein the portions are collected and automatically provided for NMR analysis and may undergo other preparation steps such as dilution, dissolution or desolvation into a deuterated solvent and provision to a probe adapted for NMR. The sample preparation and adaptation steps may be performed by the system 100 in any to the NMR spectrometer 140, mixed with a deuterated solvent to produce a sample solute suitable for NMR analysis.

The absorbent substrate portion may then be removed from the solute and discarded, and the solute analyzed. The remaining portion of the absorbent substrate 120 provided in the sample receiving area 110 may be subject to ionization or other suitable sample preparation techniques for delivery to the mass spectrometer 130, which ionization may be carried out concurrently with the preparation of the punched-out portion for NMR analysis. Accordingly, overall sample analysis time is reduced as several analytical techniques may be accomplished in parallel or in sequence in an automated fashion.

The system 100 may be configured to extract at least a portion of the sample 115, for example by transferring at least a portion of the sample 115 from an absorbed state on the absorbent substrate to a dissolved or suspended state. The portion of the sample so dissolved or suspended may be injected into the mass spectrometer 130 for ionization. The ionization may be carried out through an interface, for example a chromatography interface or a high-throughput interface configured for ionizing a sample directly injected thereon or therethrough.

The system 100 may be configured to ionize at least a portion of the extracted sample 115 in the sample receiving area 110 using acceptable ionization means, including but not limited to a laser source, an electrospray source (ESI), means for matrix assisted laser desorption (MALDI). The system 100 may further comprise means for directing an ionized portion of the sample 115 to the mass spectrometer 130, for example a funnel-shaped or frustoconical body defining a conduit between the sample receiving area 110 and the mass spectrometer 130 for permitting passage of the ionized portion to the mass spectrometer.

The system 100 may comprise further means for analyzing the sample. In non-limiting embodiments, the system 100 may comprise NMR spectroscopy means, means for performing chromatographic separation for further analysis of the eluate, including but not limited to gas chromatography or liquid chromatography, spectroscopy analytical means, for example means for performing infrared or Raman spectroscopy, and/or additional mass spectrometers and/or additional means for causing at least a portion of the sample 115 to pass from the sample 115 to a liquid or gaseous phase for further passage to an analytical means. For example, the system 100 may be configured to cause both desolvation of the analyzed sample 115 and a direct ionization of a portion of the sample 115, for further analysis/detection by a mass spectrometer. In such an embodiment, the system 100 may comprise gases, heating means, voltages means and/or means for delivering a solvent in the sample receiving area 110 or elsewhere, as well as acceptable ionization means. Accordingly, the sample 115 may be analyzed having undergone solvent extraction, desolvation and/or nebulization through the application of gases, solvents, heat and/or voltages, while another portion of the sample 115 may be analyzed having been directly ionized or directly infused.

In some embodiments, the system 100 may be configured for performing at least mass spectrometry, for example using mass spectrometer 130, and NMR analysis using an NMR spectrometer 140. Accordingly, a portion of the sample 115 may be ionized or otherwise suitably caused to enter mass spectrometer 130, and a portion of the sample may be suitably prepared for NMR analysis. For example, if the sample 115 is a liquid sample, a portion of the sample 115 may be collected from a sample container (not shown) in the sample receiving area 110 using automated collection means or not, for example an automated pipette (not shown) comprised within system 100, and further delivered to NMR spectrometer 140, which may comprise a sample preparation area 141. The portion of the sample 115 so collected may be diluted, mixed or solubilized in a suitable NMR solvent, for example a deuterated solvent, for example chloroform-D, acetone-D6, benzene-D6, deuterium oxide (D2O), DMSO-D6, ethanol-D6, and methanol-D4. It will be understood that all advantageous configurations of NMR spectrometers and their components, whether automated or not, are within the scope of this disclosure.

The system 100 may be configured to separate portions of the sample 115 before the sample 115 undergoes analysis. For example, the sample receiving area 110 may comprise means for separating a liquid sample, for example an automated pipette for collecting at least a portion of the sample 115 and transferring the portion to a suitable tube, vial, vessel or receptacle for further analysis. In other embodiments, the sample 115 may comprise a substrate, for example an absorbent substrate 120. Accordingly, the system 100 may be configured to mechanically separate at least a portion of the absorbent substrate 120 from the sample 115. The system may comprise separation means including but not limited to a knife, scalpel and/or a puncher (not shown).

The system 100 may be configured to perform a non-targeted analysis of the sample 115, for example a non-targeted metabolomic, proteomic or lipidomic analysis. Accordingly, the system 100 may be configured to analyze, record, process, output and/or transmit a metabolic profile of a sample 115, the metabolic profile comprising all compounds and/or metabolites present/detected in the sample, without prior or further filtering of the profile for known or target compounds. In embodiments, the metabolic profile of the sample 115 may comprise small molecules, amino acids, lipids, mono-, oligo- and polynucleotides, and other suspected or possible compounds whose origin, function or identification is unknown. Accordingly, the system 100 may provide a metabolic profile comprising one or more unknown, uncharacterized or partly characterized metabolites, for example metabolites forming a portion of the so-called dark metabolome.

Without being bound by any particular theory, the term “dark metabolome” as used herein refers generally to one or more metabolites that lack a full characterization. Accordingly, while such metabolites may exhibit a detectable chemical signature, their nature, origin, functions and/or correlation with other biomarkers and/or metabolites is unknown or partly known.

The system 100 may further be configured for performing imaging of a sample 115, for example a sample provided within the sample receiving area 110. The imaging may be visual imaging, IR imaging, UV imaging, or another suitable imaging technique. For example, the sample 115 may be imaged using a digital camera, for example a high-resolution digital camera 111. The system may be further configured to record, store, analyze, transmit and/or output data acquired through the imaging.

The system 100 may be configured for high-throughput analysis. For example, the system may be configured to receive the sample 115, perform mass spectrometry analysis, perform a further analytical technique such as a further spectrometry step or NMR and discard the sample 115 and/or all portions thereof in 30 seconds or less.

The system 100 may comprise means for cleaning, sterilizing and/or disinfecting the sample receiving area 110 and other components of the system. The system 100 may comprise spraying nozzles, and fluid delivery means in fluid communication with the spraying nozzles for delivering a cleaning, disinfecting or other fluid. The system 100 may comprise other cleaning and/or disinfection means, for example UV sources, gamma-ray sources, brushes, scraping blades and other means for cleaning the sample receiving area 110 and/or other components of the system 100.

The system 100 may further comprise means for receiving, storing, analyzing, outputting and/or transmitting data associated with the analytical techniques performed by the system 100. For example, the system 100 may comprise a non-transient storage medium 150, for example a memory, operatively connected to the components of the system 100, for example being operatively connected to the high-resolution digital camera 111, mass spectrometer 130 and NMR spectrometer 140. The system 100 may further comprise a processor 151, the processor being configured for causing the system 100 to action one or more of the digital camera 111, mass spectrometer 130, NMR spectrometer 140, to store data in the non-transient storage medium 150, to process the data and/or to cause the data to be outputted and/or transmitted. The non-transient storage medium 150 may comprise instructions for causing the processor to implement the actions mentioned above.

It will be understood that the non-transient storage medium 150, the processor 151 and other appropriate and/or suitable computerized components of the system 100 may be implemented as a distributed computing or cloud computing solution without departing from the present teachings.

The system 100 may comprise a processor and a non-transient storage medium, for example a processor 151 and non-transient storage medium 150, comprising instructions to implement a Machine Learning Algorithm (MLA). The MLA may be configured to access, analyze and process data comprised within the storage medium 150, for example data associated with the results of the one or more analytical techniques described above.

Referring now to FIG. 2, an exemplary method for synergistic metabolomic analysis 200 for assessing a subject's health or wellness is presented. The method comprises providing at least one sample from the subject (201), performing untargeted liquid chromatography coupled with mass spectrometry analysis on at least a portion of the sample (202), performing at least one of infrared or Raman spectroscopy, NMR spectroscopy and direct infusion mass spectrometry on at least a portion of the sample (203) and outputting a broader metabolomic profile for the subject (204).

Steps 202 and 203 may be carried out simultaneously (e.g. in parallel) or in sequence. For example, one or more portions of the sample may be separated, for example mechanically, wherein one portion may be subject to liquid chromatography coupled with mass spectrometry analysis and at least another portion may be subject to another technique.

For example, one or more portions of the sample may be subject to ionization or to another volatilization technique suitable for delivering the sample contents to a mass spectrometer, wherein a plurality of analytical equipment receive the sample contents for performing distinct analyses, for example another mass spectrometry and/or IR and/or Raman spectroscopy. In embodiments, the sample may be provided first extracted, dissolved, solubilized or diluted for one analytical technique, and subsequently provided for a second analytical technique.

For example, a sample may be solubilized or wetted using a solvent, for example a mixture of water or an alcohol or a deuterated solvent, and be further subject to analysis. It will be understood that suitable combinations of sample content extraction and/or solubilization and/or dissolution as will be apparent to ordinarily skilled persons are within the scope of the present disclosure.

The metabolomic profile outputted at 204 may comprise all compounds and/or metabolites and/or other materials present in the sample. Accordingly, the metabolomic profile may comprise identified compounds/metabolites as well as unknown compounds/metabolites, for example compounds/metabolites comprised in a subject's dark metabolome.

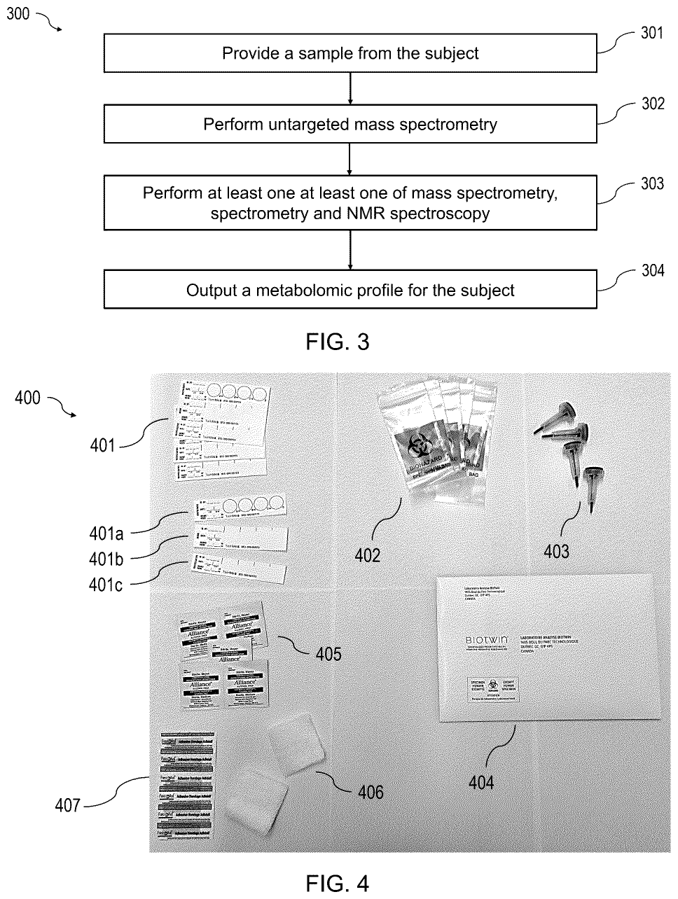

Referring now to FIG. 3, an exemplary method for synergistic metabolomic analysis 300 for assessing a subject's health or wellness is presented. The method comprises providing at least one sample from the subject (301), performing untargeted mass spectrometry analysis on at least a portion of the sample (302), performing at least one of mass spectrometry analysis, NMR spectroscopy and liquid chromatography on at least a portion of the sample (303) and outputting a metabolomic profile for the subject (304). Steps 302 and 303 may be carried out in sequence or in parallel, accordingly a system implementing the method 300 may be configured to carry out one, two or more analytical procedures at the same time.

The methods 200 and 300 may be combined, for example one or more spectroscopy and/or spectrometry steps may be carried out simultaneously and/or in parallel with NMR, chromatography, mass spectrometry and/or imaging steps. For example, a method suitably combining one or more steps of methods 200 and 300 may comprise visual, IR and/or UV imaging of a provided sample, mass spectrometry, and NMR, and optionally further analytical steps. The appropriate combinations, order and number of analytical techniques and steps will be apparent to ordinarily skilled persons practicing the present disclosure.

In embodiments, an exemplary system according to the present disclosure is configured for high-throughput blood sample analysis. An absorbent substrate comprising a plurality of blood dots is provided to the sample receiving area. An automated puncher punches out one or more of the portions of the absorbent substrate corresponding to areas where blood dots are present.

The puncher may be guided by an imaging device, for example a digital camera operatively connected to a controller, the controller being operatively connected to the puncher.

The punched out portions may be transported or otherwise provided by automated means to sample processing devices, for example to devices configured to extract, solubilize, dissolve and/or dilute the punched portions.

When the blood components comprised within the portion have been extracted, solubilized, dissolved and/or diluted at least in part, the absorbent substrate may be discarded by being transported away from the sample receiving and/or the sample preparation and/or sample processing areas, for example by being pushed by an automated blade or by being brushed away with an automated brush.

The prepared sample is then analyzed using two or more analytical techniques, for example by mass spectrometry and NMR spectroscopy.

The data associated with said analyses is provided to a non-transitory storage medium and outputted, or stored, as a metabolomic profile of the sample. The data may be associated with identifying or distinguishing features such as a user account, a user number, a patient number, or other suitable differentiating features allowing the association of data from a plurality of samples and analyses to a metabolomic profile.

The data may furthermore be associated with a particular time, for example a sample collection time and/or a sample analysis time. The metabolomic profile outputted and/or stored may accordingly comprise analytical data associated with discrete points in time. The system may be configured to track changes between metabolomic profiles associated to a subject, across two or more discrete points in time.

A processor configured to implement a machine-learning algorithm may process the data, profiles and associations described above and provide a subject or a medical professional with recommendations associated with the metabolomic profile or changes therein. For example, the recommendations may comprise grocery recommendations, such as grocery lists and recipes, or exercise recommendations, including but not limited to workout regimens, health monitoring regimens and/or recommendations to consult a physician.

Referring now to FIG. 4, an exemplary kit according to the present disclosure is presented. The kit 400 may be used for collecting a sample 115 for provision to the system 100 and/or for steps 201 and/or 301 of the methods 200 and 300 respectively. The kit 400 comprises an absorbent substrate 401. Absorbent substrate 401 may be the absorbent substrate 120 described above. In embodiments, the absorbent substrate 401 may comprise portions, such as detachable or separable strips 401a, 401b and 401c.

It will be understood that strips 401a, 401b and 401c may also be provided already separate. It will also be understood that the strips 401a, 401b and 401c may comprise different absorbent substrates or different components, fixing agents, immobilizing agents, disinfectant agents, drying agents, preserving agents and other components.

For example, strip 401a may be configured to receive a blood sample, for example one or more of blood dots, for example between one and four blood dots. Accordingly, strip 401a may comprise agents such as anti-coagulating agents, preservatives and other agents suitable for preserving the qualities and characteristics of the blood sample for analysis.

For example, strip 401b may be configured to receive a urine sample. Accordingly, strip 401b may comprise preserving, disinfectant, drying or other agents suitable for preserving the qualities and characteristics of a urine sample for analysis. For example, strip 401b may further comprise an absorbent portion and a non-absorbent portion. For example, the absorbent portion may be configured to receive the urine sample and the non-absorbent portion may be configured to be held by a user or by a health professional during collection of the sample.

For example, strip 401c may be configured for receiving a saliva sample. Accordingly, strip 401c may comprise preserving, drying, solubilizing, anti-enzymatic and other agents suitable for preserving a saliva sample. Strip 401c may further comprise flavoring or other agents for minimizing a subject's discomfort during sample collection.

Strips 401a, 401b and 401c may comprise printed indicia. The printed indicia may correspond to sample collection areas, sample identification means, and/or to input and/or recording fields for writing thereon data and/or information associated with the sample, for example collection dates and times. The sample identification means may include, but are not limited to, a sample identification number, a barcode, a QR code and other means of identifying a sample.

The kit 400 further comprises storage means for storing the samples collected using the absorbent substrate 401. For example, the kit 400 may comprise plastic bags, pockets or other receptacles 402 configured to receive one or more samples therein. For example, the receptacles 402 may be Zip-Loc™ bags. The receptacles 402 may carry indicia corresponding to their content following the use of the kit 400. For example, the indicia may correspond to a biohazard warning, or comprise handling instructions and/or indications of the content of the receptacle.

It will be understood that the receptacles 402 may be configured to be sealed using appropriate means, for example an adhesive, one or more clips, plastic or other resilient components configured to sealingly engage thereby substantially sealing the receptacle 402 and other acceptable closing and/or sealing means.

The kit 400 may further comprise blood draw means 403. The blood draw means 403 may comprise needles, lances, lancets, or other sharp or piercing features for piercing, tearing, pricking, lancing or otherwise rupturing the skin of a subject, thereby causing bleeding. The blood draw means may be provided in a sterile condition, for example as individually packaged items. The blood draw means 403 may also be sterilizable, and appropriate sterilization means and/or sterilization instructions may be provided in the kit 400. Blood draw means 403 may comprise safety features such as safety caps (not shown). Blood draw means 403 may further comprise portions or shells configured to be gripped or held by a subject and portions, for example caps or buttons, for actuating a needle or another piercing object.

Blood draw means 403 may comprise additional safety features, for example protrusions or other means for stopping the actuation of the sharp object, thereby reducing the risk of excessive skin penetration. Blood draw means 403 may further comprise resilient means for exerting a force in a direction opposite to the actuation direction of the needle or other sharp object, for example a spring or a body comprising resilient material. Suitable designs and variants of the blood draw means 403 will be apparent to skilled persons practicing the present disclosure.

The blood draw means 403 may be configured to draw capillary blood, for example by piercing, cutting or rupturing skin layers close to the skin surface. It will be understood that blood draw means 403 may be adapted for the collection of venous blood.

The kit 400 may further comprise an envelope 404. The envelope 404 may be sized for receiving receptacle 402, for example Zip-Loc™ bags, comprising samples collected on strips 401a, 401b and/or 401c. The envelope 404 may comprise suitable materials for mailing and/or otherwise transporting biosamples. For example, envelope 101 may comprise impermeabilized, reinforced or otherwise strengthened paper or cardboard for reducing the risk of sample leaks, loss, corruption and/or destruction. The envelope 404 may comprise printed indicia corresponding to at least one of a destination address, instructions for using the kit 400, instructions for filling and sealing the envelope 404, markings and/or warnings corresponding to the envelope contents following the use of the kit 400, and others.

The kit 400 may further comprise accessories for increasing the subject's safety, sterility and subject comfort. For example, the kit 400 may comprise one or more disinfecting wipes 405. The disinfecting wipes 405 may be individually packed. The kit 400 may further comprise one or more lengths of gauze 406 and one or more bandages 407, such as adhesive bandages. It will be understood that kit 400 may comprise other components, including but not limited to topical anesthetics, serviettes, reagents, holders or other means configured to receive a strip 403a-403c for drying, and other suitable components without departing from the present teachings.

The systems and methods provided herein present one or more advantages over existing techniques.

An advantage provided by the present disclosure comprises improved detection, screening and/or monitoring of a subject's wellness. For example, the principles disclosed herein provide improved sampling and detection of markers such as, but not limited to, urobilinogen, glucose, myoglobin, troponin and/or brain natriuretic peptide in blood, urine and/or saliva samples. The detection, change and/or levels of the biomarker may be correlated to conditions and/or diseases including, but not limited to hepatitis, cirrhosis, anemia, diabetes, Epstein-Barr, cytomegalovirus, bacterial conditions, neurodegenerative diseases, cardiovascular diseases and others.

A further advantage provided by the present disclosure comprises simplified patient and/or subject compliance with a health and/or wellness monitoring or screening regime. The sample collection and provision means provided herein reduce the invasiveness and/or inconvenience of sample collection, allowing a subject and/or a patient to more easily collect and provide samples of fluids such as blood, urine and/or saliva for health and/or wellness monitoring. Easier sample collection furthermore removes physical and/or psychological barriers to more frequent sampling. An advantage of such barrier removal comprises allowing a patient and/or a subject to monitor their health and/or wellness on a more regular basis and providing patient and/or subject data having a higher granularity in time.

A further advantage of the present disclosure comprises improved analytical techniques providing improved result precision. The systems provided herein allow for increased automation of sample preparation and analysis, including blood samples, urine samples and/or saliva samples. Accordingly, the need for human intervention and the possibility of human error or influence is reduced. The reliability, reproducibility and compliance of collected data with analytical and clinical standards is improved.

A further advantage of the present disclosure comprises new methods and systems for high-throughput sample processing and analysis. Reduced needs for operator intervention and improved simultaneous and/or automated analysis techniques provide for rapid generation of useful data from one or more provided samples.

A further advantage of the present disclosure comprises synergistic analysis of one or more samples, for example blood samples, urine samples and/or saliva samples. The systems and methods disclosed herein provide improved precision in the identifying and/or differentiating compounds, for example metabolites, present in one or more samples. For example, some compounds or metabolites may exhibit similar or identical spectra in a given spectrum range, for example similar or identical IR spectra. Other compounds, for example metabolites, may exhibit similar or identical NMR spectra. Accordingly, a streamlined system or method for analyzing a sample combining two or more analytical techniques provides a synergistic effect wherein compounds such as metabolites exhibiting identities or similarities according to one technique may be differentiated according to at least a second analytical technique in an automated manner, and vice versa. Increased detection breadth provides an improved metabolomic profile, allowing subjects and/or medical professionals to tailor recommendations, prescriptions, treatments, nutrition, exercise and other regimens to individual needs.

A further advantage of the present disclosure comprises improved ability to screen for and/or diagnose oral cancer, fungi, viruses (for example Epstein-Barr, cytomegalovirus) and bacteria conditions, neurodegenerative diseases and cardiovascular diseases. For example, the principles disclosed herein may be applied for screening for conditions by detecting and/or measuring myoglobin, troponin and/or brain natriuretic peptide in one or more of blood, urine and saliva samples.

The examples disclosed herein are meant to be illustrative and do not limit the scope of the present disclosure. Other useful embodiments and applications of the principles disclosed herein may be apparent to skilled persons without departing from the present teachings.

1. A system for non-targeted analysis of a metabolome in a sample, comprising:
a sample receiving area for receiving at least one sample from a subject, at least one mass spectrometer for performing at least one mass spectrometry, operatively connected to the sample receiving area for receiving at least a portion of the at least one sample therefrom;
means to further perform at least one of spectroscopy, high-throughput mass spectrometry and nuclear magnetic resonance (NMR), operatively connected to the sample receiving area for receiving at least a portion of the at least one sample therefrom;
wherein the at least one mass spectrometry and at least one of spectroscopy, high-throughput mass spectrometry and NMR identify a plurality of metabolites corresponding to the subject's metabolome; and
wherein the system is configured to output a metabolomic profile of the subject comprising all detected metabolites.

2. The system according to claim 1, wherein the system is configured to analyze a plurality of samples.

3. The system according to claim 2, wherein the system is configured to analyze the plurality of samples in parallel.

4. The system according to claim 2, wherein the system is configured to analyze the plurality of samples in sequence.

5. The system according to claim 1, wherein the at least one sample is provided on an absorbent substrate.

6. The system according to claim 5, further comprising mechanical separation means for mechanically separating at least a portion of the absorbent substrate.

7. The system according to claim 1, further comprising solvent provision means for providing a solvent to extract, solubilize, dissolve, or dilute at least a portion of the at least one sample.

8. The system according to claim 1, wherein the at least one sample comprises a urine sample.

9. The system according to claim 1, wherein the at least one sample comprises a blood sample.

10. The system according to claim 9, wherein the blood sample is a capillary blood sample.

11. The system according to claim 1, wherein the at least one sample comprises a saliva sample.

12. A synergistic metabolomic analytical method for assessing a subject's health or wellness, the method comprising:
a) providing at least one sample from the subject;
b) performing untargeted mass spectrometry on at least a portion of the at least one sample;
c) performing at least one untargeted analytical technique selected from the group consisting of: spectroscopy, NMR, another mass spectrometry on the at least one sample;
d) outputting a metabolomic profile for the subject.

13. The synergistic metabolomic analytical method according to claim 12, wherein the outputting the metabolomic profile comprises outputting a dark metabolome profile for the subject.

14. The synergistic metabolomic analytical method according to claim 12, further comprising storing at least one result from each of steps b) and c) in a non-transitory storage medium and applying a Machine Learning Algorithm to the at least one result so stored.

15. The synergistic metabolomic analytical method according to claim 14, further comprising issuing, to the subject, based on the application of the Machine Learning Algorithm to the at least one result, a recommendation with respect to at least one of nutrition, exercise, prescription medications and an appointment with a physician.

16. A kit for collecting at least one sample from a subject, comprising:
at least one absorbent substrate, the substrate being configured to receive the sample from the subject;
storage means for separately storing each absorbent substrate having received the sample; and
an envelope configured to receive the storage means, the envelope meeting requirements for biological or medical material mailing.

17. The kit according to claim 16, further comprising at least one of: blood drawing means and disinfection means.

18. The kit according to claim 17, wherein the blood drawing means is a sterile lancet.

19. The kit according to claim 17, wherein the disinfection means comprise at least one disinfectant wipe.
